(19) United States
(12) Patent Application Publication
(10) Pub. No.: US 2002/0177738 A1
(43) Pub. Date: Nov. 28, 2002

(54) Novel optically active benzylalkylcarbinols, their preparation and use

(76) Inventors: Jay F. Larrow, Wakefield, MA (US); Roger B. Pettman, Weston, MA (US); Richard A. Silva, Foxborough, MA (US); Isabelle Storet, Les Eparres (FR)

Correspondence Address: KEVIN E. MC VEIGH, RHODIA INC., 259 PROSPECT PLAINS ROAD, CRANBURY, NJ, 08512, US

(21) Appl. No.: 10/155,204

(22) Filed: May 23, 2002

Related U.S. Application Data
(60) Provisional application No. 60/293,408, filed on May 24, 2001.

(51) Int. Cl. C07C 43/235; C07C 29/44

(52) U.S. Cl. 568/662; 568/814

(57) ABSTRACT

The present invention relates to novel optically active benzylalkylcarbinols, to their preparation and to their use, in particular in perfumery.

The present invention relates to novel optically active benzylalkylcarbinols, to their preparation and to their use, in particular in perfumery.

The invention also relates to a process for their preparation.

In particular, the present invention is aimed at their use in perfumery. The compounds have advantageous olfactive properties and can, inter alia, be used to prepare perfuming compositions and perfumed products.

The perfume industry is constantly seeking products with a fragrance with an originality, volume and power that can endow the compositions in which they are contained with a unique character.

It has now been discovered that benzylalkylcarbinols in the optically active form as defined below have original olfactive properties. It should be noted that it is impossible for the skilled person to predict whether a given chemical compound will or will not possess an odour that is advantageous from the olfactive viewpoint, and also to predict its note.

The invention concerns novel optically active benzylalkylcarbinols in the (R) or (S) form with formula:

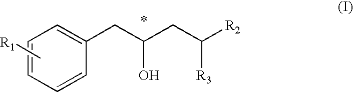

where:

R₁ represents a hydrogen atom or a linear or branched alkyl or alkoxy group containing 1 to 4 carbon atoms;

R₂ and R₃, which may be identical or different, represent a hydrogen atom or a linear or branched alkyl group containing 1 to 4 carbon atoms.

The preferred compound of the invention has formula (I) where R₁ represents a hydrogen atom and R₂ and R₃ represent a methyl group.

The invention concerns optically active benzylisobutylcarbinols.

It has been discovered that benzylisobutylcarbinol has a different odour depending on its optically active form.

In its (S) form, benzylisobutylcarbinol has a particularly interesting floral-green, mimosa powdery note.

The (R) form has a less natural green rose note.

Compounds with formula (I) can be prepared in different manners.

One route consists of reacting:

an optically active epoxy type reactant with formula (II):

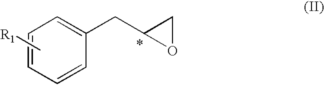

in which formula (II):

R₁ represents a hydrogen atom or a linear or branched alkyl or alkoxy group containing 1 to 4 carbon atoms;

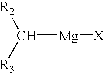

and a magnesia type reactant with formula (III):

in which formula (III):

R₂ and R₃, which may be identical or different, represent a hydrogen atom or a linear or branched alkyl group containing 1 to 4 carbon atoms.

The ratio between the number of moles of magnesia reactant and the number of moles of chiral epoxy compound is advantageously in the range 1 to 1.2.

The reaction is carried out in the presence of a conventional catalyst, such as copper iodide or iodine.

The quantity of catalyst, expressed with respect to the magnesia reactant, represents 1 mole % to 10 mole %.

The reaction is advantageously carried out at a temperature in the range −70° C. to 0° C.

At the end of the reaction, an aqueous ammonium chloride solution is added to stop the reaction.

The aqueous and organic phases are separated.

A conventional treatment is then carried out: washing the organic phase with brine (NaCl), then concentration of the organic phase.

The optically active benzylalkylcarbinol is then recovered from the organic phase obtained conventionally, for example by distillation.

It should be noted that the product of particular interest, (S)-benzylisobutylcarbinol, is obtained by reacting the magnesia reactant with (R)-(2,3-epoxypropyl)benzene.

The process of the invention uses a compound with formula (II), which can be obtained by reacting meta-chlorobenzoic acid with the unsaturated compound corresponding to formula (IV):

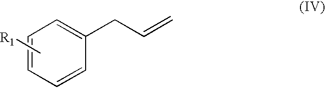

in which formula (IV):

R₁ represents a hydrogen atom or a linear or branched alkyl or alkoxy group containing 1 to 4 carbon atoms;

The preparation method to produce the racemic epoxide is known and has been described in particular by Jerry MARCH in “Advanced Organic Chemistry”, 4^{th} edition, John Wiley & Sons, 1992, p. 823.

The reaction is carried out at a temperature of 25° C. to 50° C., in an organic solvent such as an aliphatic hydrocarbon, preferably halogenated, such as chloroform, dichloromethane or dichloroethane.

At the end of the reaction, filtering is carried out to eliminate the solid residues, then extraction using a suitable organic solvent, for example tert-butyl ether.

After washing the organic phase and concentrating, the epoxy compound obtained is recovered conventionally, for example by distillation.

The epoxy compound obtained has formula (II), but is in the racemic form.

In a subsequent step, the racemic epoxide mixture is split by kinetic resolution by hydrolysis to produce one enantiomer in the form of a di-alcohol and the other enantiomer in the epoxy form.

To this end, the racemic epoxy mixture is reacted with a nucleophile such as water in the presence of a optically active catalyst.

The catalyst has been described in United States patents U.S. Pat. No. 5,665,890 and U.S. Pat. No. 5,929,232.

Preferably, it is a complex between a transition metal, preferably Cr, Mn, V, Fe, Mo, W, Ru, Ni or Co, and the “Salen” ligand with the following formula:

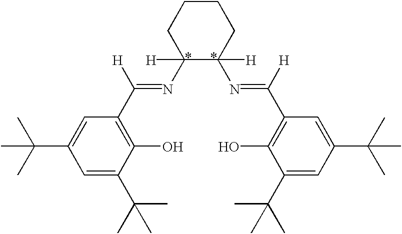

The catalyst is prepared using a transition metal, preferably cobalt and the ligand defined above obtained using the procedure described in Example 4 of U.S. Pat. No. 5,665,890.

It is also possible to use the ligands described in U.S. Pat. No. 5,665,890 in which the cyclohexane-1,2-diyl is replaced by structures:

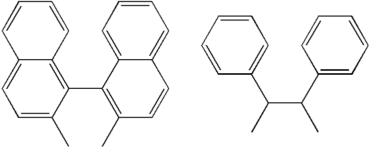

Preferably, it is (R,R)-Cosalen which is prepared using a cobalt salt and the ligand (R,R)-[1,2-di-tert-butylsalicylideamino)cyclohexane.

In the process of the invention, the racemic epoxy mixture is resolved by carrying out hydrolysis in the presence of the catalysts described above.

The reaction is carried out between −20° C. and 50° C., preferably in the range 0° C. to ambient temperature (usually in the range 15° C. to 25° C.).

It is advantageously carried out in a solvent preferably selected from tert-butylmethylether, ethyl ether or tetrahydrofuran.

In one implementation of the invention, water is slowly added to the reaction medium comprising the racemic epoxide; the catalyst is the organic solvent.

The chiral epoxide with formula (II) is recovered conventionally, for example by distillation.

The present invention also concerns perfuming compositions, products and substances characterized in that they comprise an effective quantity of at least one benzylalkylcarbinol with formula (I) as an active principle having an influence on the odour.

Compounds with formula (I) give off a very interesting scent.

These products can be used as perfuming ingredients in perfuming compositions, substances and perfumed products.

The term “perfuming compositions” means mixtures of various ingredients such as solvents, solid or liquid supports, fixatives, various scenting compounds, etc . . . , into which benzylalkylcarbinols with formula (I) are incorporated, which are used to produce a variety of types of finished products, with the desired fragrance.

Perfume bases constitute preferred examples of perfuming compositions in which the benzylalkylcarbinols with formula (I) can advantageously be used.

Eau de toilette, after-shave lotion, perfume, soap, bath or shower gel or deodorant or antiperspirant in the form of sticks or lotions constitute examples of finished products or substances which the benzylalkylcarbinols with formula (I) endow with their original note.

They can also be used in all types of shampoos and hair-care products.

They can also perfume all types of talcs or powders.

They can also be used in room sprays or any cleaning product.

A further example of compositions in which the compounds can advantageously be used is represented by the usual detergent compositions. Such compositions generally comprise one or more of the following ingredients: anionic, cationic or amphoteric surfactants, bleaching agents, optical brighteners, various fillers, and anti-redepositing agents. The nature of these various components is not critical and the benzylalkylcarbinols, with formula (I) can be added to any type of detergent composition. They can be introduced into fabric softeners, in liquid form or into compositions deposited on a support, usually a non-woven support, for use in clothes dryers.

The amount of the compositions of the invention of benzylalkylcarbinol, with formula (I), expressed as the percentage by weight in the composition under consideration, depends on the nature of the composition (a base for a perfume or eau de toilette, for example) and the strength and nature of the desired influence in the finished product. It is clear that in a perfume base the quantity of benzylalkylcarbinol with formula (I) can be very high, for example over 50% by weight, and can attain 90% by weight while in a perfume, an eau de toilette or an after-shave lotion, this quantity can be below 50% by weight.

In detergent compositions, in particular for domestic use, and in soaps, the quantity of benzylalkylcarbinol can be of the order of 1% to 2%.

It can also be used in perfumed shampoos in an amount of 0.5% to 2%, or to perfume any hair product.

Thus the lower limit of the amount of benzylalkylcarbinol with formula (I) can be that which causes a perceptible modification in the scent or fragrance or the note of the finished product. In some cases, this minimum amount can be of the order of 0.01% by weight. Clearly, quantities which are not included in the limits indicated above can be employed without departing from the scope of the invention.

Examples of implementations of the invention will now be given.

### EXAMPLE

### 1. In this example, (S)-benzylisobutylcarbinol was produced.

It was prepared by reacting of isopropylmagnesium chloride with(R)-(2,3-epoxypropyl)benzene obtained from allylbenzene).

Preparation of (2,3-epoxypropyl)benzene.

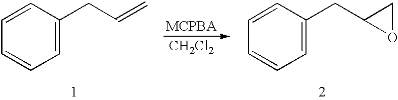

A 12 L four necked round bottom flask was equipped with a mechanical stirrer.

The flask was charged with allyl benzene (472 g, 4.0 mol) and dichloromethane (1 L) m-chloroperbenzoic acid (500 g, 2.9 mol) in dichloromethane (3 L) was added to the mixture in portions over 1.5 h (internal temperature kept below 35° C., the initial exotherm was controlled with an ice bath).

A slurry of m-chloroperbenzoic acid (400 g, 2.3 mol) in dichloromethane (3 L) was added to the reaction in portions over 1 h.

The reaction was allowed to warm to room temperature and m-chloroperbenzoic acid (114 g, 0.66 mol) was added to the reaction as a solid. The reaction was left stirring overnight.

The following morning GC analysis showed the reaction was 98% complete.

To drive the reaction to completion m-chloroperbenzoic acid (50 g , 0.29 mol) was added to the reaction as a solid.

After 5 h of stirring the reaction showed a conversion of 99.1% by GC.

The reaction was filtered to remove solids and the filtrate was concentrated under reduced pressure.

Additional solid formed upon concentration and was removed by filtration.

Tert-Butyl methyl ether (1 L) was added to the filtrate.

The solution was washed with a NaHCO₃ solution (4×300 mL), a Na₂SO₃ solution (50 g/300 mL H₂O), and then a Na₂SO₃ solution (80 g/300 mL H₂O) by stirring for 1.5 h.

The organic layer was separated, dried over Na₂SO₄ and concentrated under reduced pressure.

Vacuum distillation afforded 402.0 g of (2,3-epoxypropyl)benzene 2 for an isolated yield of 75%.

Preparation of (R)-(2,3-epoxypropyl)benzene.

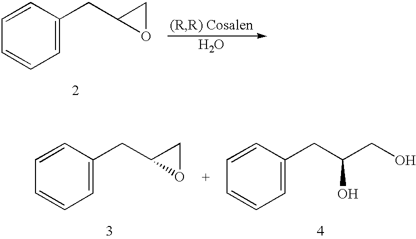

A 250 mL three necked round bottom flask was fitted with a mechanical stirrer.

To the flask was added (R,R) Jacobsen Cobalt Catalyst (1.1 g, 1.9 mmol), (2,3-epoxypropyl)benzene 2 (50 g, 0.37 mol), acetic acid (0.46 g, 7.6 mmol), and tetrahyrofuran (3.9 mL, 3.5 g, 49 mmol).

The mixture was cooled in an ice bath as water (3.6 mL, 0.20 mol) was added slowly.

The reaction mixture was allowed to warm to room temperature and was left to stir overnight.

The following morning chiral GC analysis showed the reaction to be 95.6% complete.

To drive the reaction to completion water (0.32 mL, 0.02 mol) was added.

After 2 h of stirring, chiral GC analysis showed the reaction to be 97.6% complete.

The reaction was distilled at 1 torr and the product was collected at 55° C. (R)-(2,3-epoxypropyl)benzene 3 (98% ee, >99% chemical purity by GC) weighed 15 g for an isolated yield of 30%.

Preparation of (S)-benzylisobutylcarbinol.

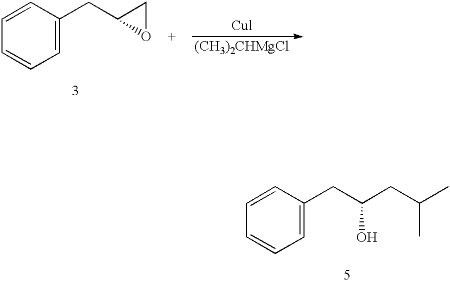

A dried 250 mL three necked round bottom flask was equipped with a mechanical stirrer, thermocouple, and a Claisen adapter fitted with a dropping funnel and rubber septum.

To the flask was added Copper (I) iodide (1.0 g, 5.5 mmol).

The flask was flushed with nitrogen and a nitrogen atmosphere was maintained thought the remaining steps.

A 2.0 M solution of isopropyl magnesium chloride in tetrahydrofuran (65 mL, 13,7 g, 0.13 mol) was added and the mixture was stirred at room temperature for 0.5 h.

The mixture was cooled to −60° C. in a dry ice/IPA bath. (R)-(2,3-epoxypropyl)benzene 3 (15.0 g, 0.11 mol) in anhydrous tetrahydrofuran (60 mL) was added drop wise slowly to avoid exotherm.

Following the completion of addition the reaction mixture was allowed to stir at −60° C. for 0.5 h.

The reaction mixture was slowly allowed to warm to room temperature. A latent exotherm was noted.

The reaction mixture was cooled in an ice bath and the reaction was quenched with a 25% solution of NH₄Cl in water.

The mixture was poured into a separatory funnel.

Additional water was added and the product was extracted with ethyl acetate (2×).

The combined organic layers were washed with brine, dried with Na₂SO₄, and concentrated.

The crude product weighed 19.1 g.

The product was distilled at 1 torr and collected at 83° C.

(S)-Benzylisobutylcarbinol 5 (97.8% purity by GC) weighed 14.0 g for a isolated yield of 72%.

2. The obtained (S)-benzylisobutylcarbinol has a particularly interesting floral-green, mimosa powdery note, with a very natural effect.

1. Novel optically active benzylalkylcarbinols in the (R) or (S) form with formula: 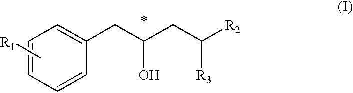
where:
R₁ represents a hydrogen atom or a linear or branched alkyl or alkoxy group containing 1 to 4 carbon atoms;
R₂ and R₃, which may be identical or different, represent a hydrogen atom or a linear or branched alkyl group containing 1 to 4 carbon atoms.

2. Benzylalkylcarbinol according to claim 1 characterized it has formula (I) where R₁ represents a hydrogen atom and R₂ and R₃ represent a methyl group.

3. Process for the preparation of an optically active benzylalkylarbinol according to claim 1 or 2 characterized in that it consists of reacting:
an optically active epoxy type reactant with formula (II): 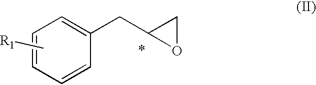
in which formula (II):
R₁ represents a hydrogen atom or a linear or branched alkyl or alkoxy group containing 1 to 4 carbon atoms; 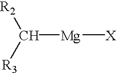
and a magnesia type reactant with formula (III):
in which formula (III):
R₂ and R₃, which may be identical or different, represent a hydrogen atom or a linear or branched alkyl group containing 1 to 4 carbon atoms.

4. Process according to claim 3 characterized in that the reaction is carried out in the presence of copper iodide or iodine catalyst.

5. Process according to claim 3 characterized in that (S)-benzylisobutylcarbinol, is obtained by reacting the magnesia reactant with (R)-(2,3-epoxypropyl)benzene.

6. An optically active epoxy type reactant with formula (II): 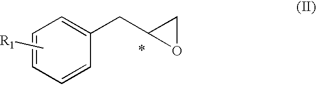
in which formula (II):
R₁ represents a hydrogen atom or a linear or branched alkyl or alkoxy group containing 1 to 4 carbon atoms.

7. Process for the preparation of an optically active type reactant with formula (II) according to claim 6 characterized in that it consists of reacting meta-chlorobenzoic acid with the unsaturated compound corresponding to formula (IV): 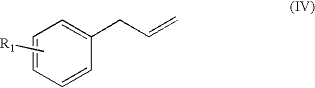
in which formula (IV):
R₁ represents a hydrogen atom or a linear or branched alkyl or alkoxy group containing 1 to 4 carbon atoms,
and in a subsequent step, splitting the racemic epoxide mixture by kinetic resolution by hydrolysis to produce one enantiomer in the form of a di-alcohol and the other enantiomer in the epoxy form.

8. Process according to claim 7 characterized in that hydrolysis is carried out with water in the presence of an optically active catalyst.

9. Process according to claim 8 characterized in that the optically catalyst is a complex between a transition metal, preferably Cr, Mn, V, Fe, Mo, W, Ru, Ni or Co, and the “Salen” ligand.

10. Process according to claim 8 characterized in that the optically catalyst is (R,R)-Cosalen.

11. A process for the production of perfuming compositions, perfumed products an d substances for perfumery characterized in that an effective quantity of an optically active benzylalkylcarbinol of formula (I) is added to the 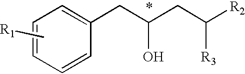
usual constituents of such compositions, substances and finished products:
where:
R₁ represents a hydrogen atom or a linear or branched alkyl or alkoxy group containing 1 to 4 carbon atoms;
R₂ and R₃, which may be identical or different, represent a hydrogen atom or a linear or branched alkyl group containing 1 to 4 carbon atoms.

12. A process according to claim 11, characterized in that the benzylalkylcarbinol is (R) or (S)-benzylisobutylcarbinol.

13. Perfuming compositions, perfumed products and substances, characterized in that they comprise an effective quantity of an optically active benzylalkylcarbinol of formula (I) as an active ingredient having an influence 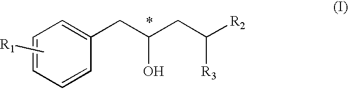
on the scent:
where:
R₁ represents a hydrogen atom or a linear or branched alkyl or alkoxy group containing 1 to 4 carbon atoms;
R₂ and R₃, which may be identical or different, represent a hydrogen atom or a linear or branched alkyl group containing 1 to 4 carbon atoms.

14. Compositions according to claim 13, characterized in that the benzylalkylcarbinol is (R) or (S)-benzylisobutylcarbinol.

15. A perfumed article according to any one of claims 13 and 14 in the form of a perfume, eau de toilette, after-shave lotion, soap, bath or shower gel, deodorising or antiperspirant product, a shampoo or any other hair-care product, talc or powder of any type, an air freshener, any cleaning product or detergent composition, or a fabric softener.

16. Use of an optically active benzylalkylcarbinol of formula (I) as a perfuming ingredient: 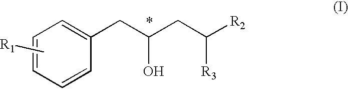
where:
R₁ represents a hydrogen atom or a linear or branched alkyl or alkoxy group containing 1 to 4 carbon atoms;
R₂ and R₃, which may be identical or different, represent a hydrogen atom or a linear or branched alkyl group containing 1 to 4 carbon atoms.

17. Use of (S)-benzylisobutylcarbinol as a particularly interesting floral-green, mimosa, powdery note.

18. Use of (R)-benzylisobutylcarbinol as a green rose note.
